(19) United States
(12) Patent Application Publication
(10) Pub. No.: US 2016/0213682 A1
(43) Pub. Date: Jul. 28, 2016

(54) FULVESTRANT COMPOSITIONS

(71) Applicants: Salah Uddin AHMED, New York, NY (US); Phanidhara Rao KOTAMRAJ, Westwood, NJ (US); Sudhir Rao GORUKANTI, Tuxedo Park, NY (US); Harish CHERIVIRALA, Tuxedo Park, NY (US); Ramesh SRIRANGAM, New Milford, NJ (US); Alok Brindawanlal NAMDEO, Hyderabad (IN); Venkata Sesha Kanthikiran VARANASI, Hyderabad (IN)

(72) Inventors: Salah Uddin AHMED, New York City, NY (US); Phanidhara Rao KOTAMRAJ, Westwood, NJ (US); Sudhir Rao GORUKANTI, Tuxedo Park, NY (US); Harish CHERIVIRALA, Tuxedo Park, NY (US); Ramesh SRIRANGAM, New Milford, NJ (US); Alok Brindawanlal NAMDEO, Hyderabad (IN); Venkata Sesha Kanthikiran VARANASI, Hyderabad (IN)

(21) Appl. No.: 14/917,108

(22) PCT Filed: Sep. 5, 2014
(86) PCT No.: PCT/IB2014/064282

§ 371 Date: Mar. 7, 2016

Related U.S. Application Data
(60) Provisional application No. 61/874,557, filed on Sep. 6, 2013.

(51) Int. Cl. A61K 31/565; A61K 47/10; A61K 47/12; A61K 9/00; A61K 47/44

(52) U.S. Cl. CPC A61K 31/565; A61K 9/0019; A61K 47/44; A61K 47/12; A61K 47/10

(57) ABSTRACT

The invention relates to novel formulations of fulvestrant for intramuscular administration. Methods of preparing such fulvestrant formulations are also provided. The present invention further relates to the use of fulvestrant formulation in the treatment of a disease or condition that is or is believed to be responsive to anti-estrogen therapy, such as cancer.

### FIELD OF THE INVENTION

The present invention relates to novel formulations of fulvestrant for intramuscular administration. Methods of preparing such fulvestrant formulations are also provided. The present invention further relates to the use of fulvestrant formulation in the treatment of a disease or condition that is or is believed to be responsive to anti-estrogen therapy, such as cancer.

### BACKGROUND OF THE INVENTION

Fulvestrant (7-alpha-[9-(4,4,5,5,5-penta fluoropentylsulphinyl)nonyl]estra-1,3,5-(10)-triene-3,17-beta-diol) is a new class of anti-oestrogen described by the term oestrogen receptor (ER) downregulator. The molecular structure is shown by formula (I):

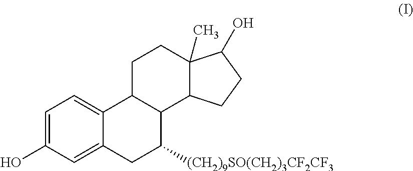

Fulvestrant is an ER antagonist that binds to ER in a competitive manner and down regulates the ER protein in human breast cancer cells leading to the inhibition of estrogen stimulated tumor growth. Commercially available FASLODEX® is an intramuscular injection of 250 mg (50 mg/ml) fulvestrant in a sterile oily solution provided in a single 5 ml pre-filled syringe, as long acting injection(s) and is indicated for the treatment of hormone receptor positive metastatic breast cancer in postmenopausal women with disease progression following anti-estrogen therapy. The recommended dosage is 500 mg administered intramuscularly into buttocks slowly as two 5 ml injections, one in each buttock, on day 1, 15, 29 and once monthly thereafter. Upon intramuscular injection of 500 mg dose followed by a second dose after two weeks allows the steady state to be reached in a month. Apparently, the formulation forms a depot intramuscularly, releasing the drug over a prolonged period of time to meet the dosing regimen.

Fulvestrant formulations are administered intramuscularly. Intramuscular formulations need to have a high concentration of active as only relatively low volumes can be injected. Fulvestrant has a low solubility in many pharmaceutically acceptable solvents due to its lipophilicity and accordingly is difficult to formulate at appropriate concentrations. Formulations comprising 50 mg fulvestrant, 400 mg benzyl alcohol and castor oil are described in U.S. Pat. No. 5,183,814. However, it is stated in WO 01/51056 that the high proportion of alcohol required for this formulation may be undesirable for large scale manufacturing processes. In addition, it is well known that a high level of alcohol in a formulation for intramuscular injection increases the pain experienced by the subject after injection.

WO 01/51056 further discloses fulvestrant formulations comprising castor oil together with an alcohol and benzyl benzoate. The alcohol and non-aqueous ester solvent are claimed to increase the overall solubility of the fulvestrant and to decrease the volume of alcohol utilized in the formulation so that the administration volume is therapeutically acceptable. The addition of benzyl benzoate is taught as essential in achieving a fulvestrant formulation of at least 45 mg/ml and a total formulation volume of 6 ml or less.

There are several other patents in state of the art which disclose benzyl benzoate as the essential constituent for oily parenteral composition for intramuscular administration.

GB 1207571 discloses oily injectable composition for hormone administration, comprising admixture of benzyl benzoate, chlorobutanol and vegetable oil as vehicle.

GB 1569286 discloses oily depot solutions of gestagens for intramuscular injection; the preferred vehicle comprises castor oil/benzyl benzoate.

GB 1126892 discloses a medicinal preparation of progesterone in an oily solvent comprising castor oil/benzyl benzoate for intramuscular injection in the treatment of hypertrophic condition of the prostate.

US 20090227552 discloses an alternate formulation of fulvestrant comprising propylene glycol/polyethylene glycol thereby avoiding a non-aqueous ester solvent.

In an attempt to develop novel fulvestrant formulations, the present inventors have surprisingly found that use of benzoic acid resulted in a fulvestrant formulation having sufficiently high concentration to be used intramuscularly. These novel formulations provide satisfactory release of fulvestrant over an extended period of time. Surprisingly, benzoic acid despite its lower log P (1.87) compared to benzyl benzoate (3.97) was found to favor the bioavailability of the highly lipophilic fulvestrant and thus providing the requisite sustained release property to the formulations. Other advantages of benzoic acid include ease of handling & manufacturing (being solid in nature and being easily soluble in alcohol and oil), a lower toxicity potential and fewer metabolites as compared to benzyl benzoate. To our knowledge, none of the prior art discloses the use of benzoic acid in a fulvestrant formulation for intramuscular administration.

### SUMMARY OF THE INVENTION

The present specification relates to a novel formulation of fulvestrant comprising benzoic acid.

In one aspect, the present specification relates to a pharmaceutical formulation comprising:

(i) fulvestrant or a pharmaceutically acceptable salt thereof;
(ii) benzoic acid;
(iii) one or more alcohols; and
(iv) one or more vegetable oils.

In another aspect, the present specification relates to a pharmaceutical formulation comprising:

(i) fulvestrant or a pharmaceutically acceptable salt thereof;
(ii) benzoic acid;
(iii) ethanol;
(iv) benzyl alcohol;
(v) castor oil; and
(vi) sesame oil.

In another aspect, the present specification relates to a pharmaceutical formulation comprising:

(i) 2-10% w/v fulvestrant or a pharmaceutically acceptable salt thereof;
(ii) 1-15% w/v benzoic acid
(iii) 2-40% w/v of one or more alcohols; and
(iv) at least 55% w/v castor oil.

In another aspect, the present specification relates to a pharmaceutical formulation comprising:

(i) 5% w/v fulvestrant or a pharmaceutically acceptable salt thereof;
(ii) 4% w/v benzoic acid;
(iii) 10% w/v benzyl alcohol;
(iv) 10% w/v ethanol; and
(v) 55-70% w/v castor oil.

In another aspect, the present specification relates to a pharmaceutical formulation comprising:

(i) 2-10% w/v fulvestrant or a pharmaceutically acceptable salt thereof;
(ii) 1-15% w/v benzoic acid;
(iii) 2-20% w/v benzyl alcohol;
(iv) 2-20% w/v ethanol;
(v) at least 40% w/v castor oil; and
(vi) at least 1% w/v sesame oil.

In another aspect, the present specification relates to a pharmaceutical formulation comprising:

(i) 5% w/v fulvestrant or a pharmaceutically acceptable salt thereof;
(ii) 2-8% w/v benzoic acid;
(iii) 2-20% w/v benzyl alcohol;
(iv) 2-20% w/v ethanol;
(v) 55-70% w/v castor oil; and
(vi) 2-12% w/v sesame oil.

In another aspect, the present specification relates to a pharmaceutical formulation comprising:

(i) 5% w/v fulvestrant or a pharmaceutically acceptable salt thereof;
(ii) 6% w/v benzoic acid;
(iii) 10% w/v benzyl alcohol;
(iv) 10% w/v ethanol;
(v) 60% w/v castor oil; and
(vi) 9% w/v sesame oil.

In yet another aspect, the formulation of present specification relates to a method for the treatment of hormone receptor positive tumors.

The formulation of the present specification is administered parenteraly, e.g. intramuscularly.

In yet another aspect, the present specification relates to a pharmaceutical formulation of fulvestrant which is bioequivalent to the currently marketed fulvestrant formulation FASLODEX®.

### BRIEF DESCRIPTION OF FIGURE

FIG. 1: Time-Concentration Plot of different formulations of fulvestrant after single dose administration in male Wistar rats

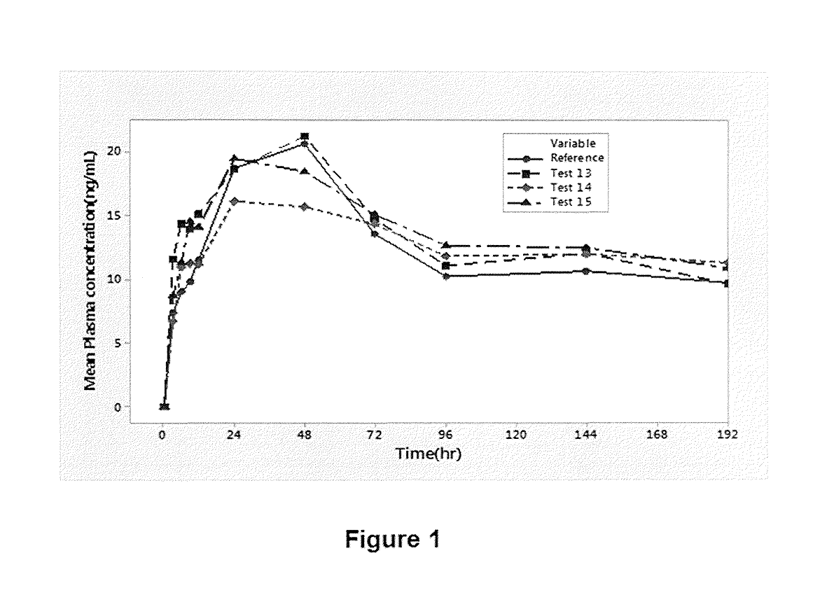

### DESCRIPTION OF THE INVENTION

The present specification relates to a novel formulation of fulvestrant comprising benzoic acid. The formulation is substantially free of benzyl benzoate.

In one aspect, the present specification relates to a pharmaceutical formulation comprising:

(i) fulvestrant or a pharmaceutically acceptable salt thereof;
(ii) benzoic acid;
(iii) one or more alcohols; and
(iv) one or more vegetable oils.

In another aspect, the present specification relates to a pharmaceutical formulation comprising:

(i) fulvestrant or a pharmaceutically acceptable salt thereof;
(ii) benzoic acid;
(iii) ethanol;
(iv) benzyl alcohol;
(v) castor oil; and
(vi) sesame oil.

As used herein, the term “pharmaceutical formulation” refers to the preparation of fulvestrant in a form suitable for administration to a human, wherein the pharmaceutical formulation is adapted for intramuscular administration to attain a therapeutically significant level of blood plasma fulvestrant concentration.

In another aspect, the present specification relates to a pharmaceutical formulation comprising:

(i) 5% w/v fulvestrant or a pharmaceutically acceptable salt thereof;
(ii) 2-8% w/v benzoic acid;
(iii) 2-20% w/v benzyl alcohol;
(iv) 2-20% w/v ethanol; and
(v) at least 55% w/v castor oil.

In another aspect, the present specification relates to a pharmaceutical formulation comprising:

(i) 2-10% w/v fulvestrant or a pharmaceutically acceptable salt thereof;
(ii) 1-15% w/v benzoic acid;
(iii) 2-20% w/v benzyl alcohol;
(iv) 2-20% w/v ethanol;
(v) at least 40% w/v castor oil; and
(vi) at least 1% w/v sesame oil.

In another aspect, the present specification relates to a pharmaceutical formulation comprising:

(i) 5% w/v fulvestrant or a pharmaceutically acceptable salt thereof;
(ii) 6% w/v benzoic acid;
(iii) 10% w/v benzyl alcohol;
(iv) 10% w/v ethanol;
(v) 60% w/v castor oil; and
(vi) 9% w/v sesame oil.

In another aspect, the present specification relates to a pharmaceutical formulation comprising comprising:

(i) 5% w/v fulvestrant or a pharmaceutically acceptable salt thereof;
(ii) 4% w/v benzoic acid;
(iii) 10% w/v benzyl alcohol;
(iv) 10% w/v ethanol;
(v) 55-70% w/v castor oil; and
(vi) 2-12% w/v sesame oil.

The concentration of fulvestrant or a pharmaceutically acceptable salt thereof is 2-10% w/v, 3-9% w/v, 4-8% w/v, 4-7% w/v, 4-6% w/v, e.g. 5% w/v.

The concentration of benzoic acid is 1-15% w/v, 2-14% w/v, 3-13% w/v, 4-12% w/v, 4-10% w/v, e.g. 4-8% w/v, e.g. 6% w/v or 5% w/v or 4% w/v.

As used herein, the term “alcohol” includes any pharmaceutically-acceptable alcohol for parenteral administration such as ethanol, benzyl alcohol or a mixture of ethanol and benzyl alcohol. The ranges of alcohol employed in the present specification are: 2-35% w/v, 2-20% w/v, 4-20% w/v, 5-20% w/v, 6-20% w/v, 7-20% w/v, 8-20% w/v, 10-20% w/v, e.g. the range of alcohol may be 10-30% w/v, 10-25% w/v, 10-20% w/v; or a mixture of 10% w/v ethanol and 10% w/v benzyl alcohol.

As used herein, the term “vegetable oil” includes any pharmaceutically-acceptable vegetable oil for parenteral administration such as castor oil, sesame oil, cottonseed oil, peanut oil, corn oil, soybean oil, olive oil or mixtures thereof. eg. a mixture of castor oil and sesame oil. The ranges of vegetable oil employed in the present specification are: 1-80% w/v, 5-75% w/v, e.g. castor oil is at least 40% w/v, 45% w/v or 50% w/v; sesame oil is at least 1% w/v, 2% w/v, 3% w/v, 4% w/v, 5% w/v, 6% w/v or quantity sufficient to make up the volume, e.g. a mixture of at least 55% w/v castor oil and at least 7% w/v sesame oil or a mixture of 55-70% w/v castor oil and 2-12% w/v sesame oil.

The present specification is not limited to the use of benzoic acid. Other agents that may aid in improving the solubility of fulvestrant may include glycerin, polyoxyethylene fatty acid ester, propylene glycol, polyethylene glycol, sodium benzoate, PEG, PEG-40, N, N-dimethyl acetamide, Miglyol 840, crodamol GTCC, captex, medium chain trigycerides or mixtures thereof.

In another aspect, formulation of the present specification may optionally comprise compatible antioxidants. Suitable non-limiting antioxidants include, without limitation, lipoic acid and its structural analogs such as dihydrolipoic acid, methionine and other sulfur-containing amino acids, acetone sodium bisulfate, propyl gallate, BHT, BHA and sodium formaldehyde sulfoxylate.

In yet another aspect, the formulation of present specification relates to a method for the treatment of hormone receptor positive tumors.

The formulation of present specification is useful for the treatment of hormone receptor positive metastatic breast cancer in postmenopausal women with disease progression following anti-estrogen therapy.

The formulation of the present specification is administered parenteraly, e.g. intramuscularly into the buttocks as two 5 ml injections, one in each buttock.

It will be understood that the attendant physician may wish to administer the intramuscular injection as a divided dose, i.e. a 5 ml formulation is sequentially administered in two separate injections of 2.5 ml, and this is a further feature of the specification.

The process for the preparation of fulvestrant formulation of the present specification may be any conventional suitable process.

In yet another aspect, the present specification relates to a pharmaceutical formulation of fulvestrant which is bioequivalent to the currently marketed fulvestrant formulation FASLODEX®.

The present inventors found that the formulations of the specification, after intra-muscular injection, may provide satisfactory release of fulvestrant over an extended period of time.

As used herein, the term “therapeutically significant levels” means that blood plasma concentrations of at least 2.5 ng/ml, at least 3 ng/ml, at least 8.5 ng/ml, and up to 12 ng/ml of fulvestrant are achieved in the patient. Preferably blood plasma levels should be less than 15 ng/ml.

As used herein, the term “extended period” refers to at least two weeks, at least three weeks, at least four weeks of continuous release of fulvestrant. eg. the extended release is achieved for 36 days, at least 2-5 weeks, 2.5-5 weeks, 2.5-4 weeks, 3-4 weeks, 3.5-4 weeks or for at least about 4 weeks.

The present inventors have developed a series of test formulations (Table 1). During experimentation it was surprisingly found that the introduction of a suitable quantity of benzoic acid in the test formulation eases the solubilisation of fulvestrant to achieve a concentration of about 50 mg/ml.

TABLE 1

| | Test | Test | Test | Test | Test | Test | Test |
|---|---|---|---|---|---|---|---|
| Ingredient | 1 | 2 | 3 | 4 | 5 | 6 | 7 |
| Fulvestrant | 5 | 5 | 5 | 5 | 5 | 5 | 5 |
| Ethanol | 10 | 10 | 10 | 10 | 10 | 10 | 10 |
| Benzyl alcohol | 10 | 10 | 10 | 10 | 10 | 10 | 10 |
| Castor oil | 60 | 60 | 60 | 60 | 58.05 | 58.05 | 58.05 |
| Benzoic acid | — | 4 | 6 | 8 | 4 | 6 | 8 |
| Sesame oil | 15 | 11 | 9 | 7 | 8.53 | 7.35 | 6.0 |
| (q.s. to 100 ml) | | | | | | | |

All values are in % w/v

The above formulations were prepared by the general process of mixing benzoic acid with ethanol and benzyl alcohol, fulvestrant was added to this solution and stirred until completely dissolved. The prepared solution is then mixed with castor oil and final weight was adjusted with sesame oil. The solution was sterilized by filtration using one or two filters of 0.2 μm porosity.

These test formulations as prepared above were subjected to suitable in vitro evaluation to understand the effect of individual excipients on properties such as solubility, solvent migration rate, precipitation kinetics (tendency to prevent the drug from precipitation or to keep the drug in solution).

It is hypothesized that for oil based formulations, higher drug holding capacity of depot tends to prevent precipitation of drug for longer periods, resulting in better bioavailability. Migration rate of alcohols from the injection site influences the initial release rate as they have the highest solubility for fulvestrant in the formulation. The later part of the sustained release is controlled by the composition left after the solvent migration. Therefore it is important to determine the amount of soluble drug in the remaining formulation during and after solvent migration. Hence keeping a track on the changing saturation solubility of the depot can help in providing a better understanding of release characteristics of such formulations.

Precipitation study is considered as a suitable in vitro evaluation tool as it will provide the following key information.

1. The capacity of the formulation to keep the drug in solution during and after solvent migration. Therefore, solubility property of the constantly changing medium may play a key role in the rate and extent of absorption.
2. The release behavior of the soluble drug in the depot is a function of its composition. The rate and extent of solvent migration dictates the composition of formulation remaining as a function of time. This factor is also believed to affect both Cmax and AUC.

### Solubility Data

Solubility studies were conducted to determine the solvating ability of fulvestrant in test formulations. Any suitable method can be used to perform solubility studies. Accurately weighed amount of fulvestrant was added to a mixture of benzoic acid, ethanol and benzyl alcohol and vertexed until a clear solution was obtained. Volume was made up to 100% with suitable solvent (castor oil/sesame oil). The final formulation was vertexed and kept on the shaker for 48 hours at room temperature. To determine the solubility, the formulation was centrifuged at 10,000 rpm for 5 minutes and the supernatant was filtered through 0.45μ filter. Accurately weighed and transferred 250 mg of filtrate in to a 25 mL volumetric flask, diluted to volume with methanol and analyzed by HPLC.

HPLC Conditions: HPLC Column: Symmetry C8,150×4.6 mm 3.5 μm, Mobile Phase: Water/ACN (400/600, v/v), Flow Rate: 1.5 mL/minute (Isocratic), Injection Volume: 100 μL, Column Temp: 40° C., Detector Wavelength: 225 nm, Run Time: About 8 min

The solubility study results are presented in Table 2.

TABLE 2

| | Solubility |
|---|---|
| Formulation | (mg/g) |
| Test 1 | 80.2 |
| Test 2 | 125.6 |
| Test 3 | 125.9 |
| Test 4 | 130.4 |

### Precipitation Kinetics:

The precipitation kinetics experiment was conducted under controlled condition by adding 1 part of formulation to 4 parts of Phosphate buffered saline solution (pH 7.4) by weight followed by agitating them on reciprocating shaker for one week. Samples were taken at regular intervals to measure the concentration of fulvestrant remaining in the oil layer. The results are available in Table 3. Similar conditions were simulated in a low volume dissolution vessel with paddles at 50 RPM to conduct the experiment under more controlled conditions. Additionally, the aqueous layer consisted of 30% w/w hydroxyl propyl beta cyclodextrin (HPBCD) to drive the partitioning of drug into aqueous layer.

TABLE 3

| | Formulation | | | | | |
|---|---|---|---|---|---|---|
| | Test 5 | | Test 6 | | Test 7 | |
| | Aq. | Oil | Aq. | Oil | Aq. | Oil |
| Time (Days) | Layer | Layer | Layer | Layer | Layer | Layer |
| 0 | 0.00 | 101 | 0.00 | 101 | 0.00 | 101 |
| 0.25 | — | 103 | — | 103 | — | — |
| 1 | 0.00 | — | 0.00 | — | 0.00 | 103 |
| 2 | 0.00 | 112 | 0.00 | 111 | 0.00 | 111 |
| 3 | 0.02 | 113 | 0.02 | 112 | 0.02 | 112 |
| 4 | 0.02 | 113 | 0.02 | 114 | 0.02 | 114 |
| 5 | 0.04 | 88 | 0.02 | 105 | 0.03 | 116 |
| 6 | 0.03 | 61 | 0.08 | 99 | 0.03 | 117 |
| 8 | 0.09 | 82 | 0.23 | 86 | 0.34 | 121 |
| 9 | 0.12 | 78 | 0.38 | 81 | 0.28 | 114 |
| 10 | 0.38 | 75 | 0.79 | 75 | 0.62 | 95 |
| 11 | 0.44 | 71 | 1.13 | 71 | 0.72 | 81 |
| 14 | 1.46 | 64 | 2.55 | 67 | 1.89 | 71 |
| 18 | 3.88 | 62 | 3.91 | 63 | 2.98 | 65 |

A second set of following formulations were prepared and subjected to in vitro and in vivo study.

TABLE 4

| | | Test | Test | Test | Test | Test | Test | Test | Test |
|---|---|---|---|---|---|---|---|---|---|
| Ingredient | Reference | 8 | 9 | 10 | 11 | 12 | 13 | 14 | 15 |
| Fulvestrant | 5 | 5 | 5 | 5 | 5 | 5 | 5 | 5 | 5 |
| Ethanol | 10 | 10 | 10 | 10 | 10 | 10 | 10 | 10 | 10 |
| Benzyl | 10 | 10 | 10 | 10 | 10 | 10 | 10 | 10 | 10 |
| alcohol | | | | | | | | | |
| Benzyl | 15 | — | — | — | — | — | — | — | — |
| Benzoate | | | | | | | | | |
| Castor oil | 57.91* | 58.05 | 58.05 | 65.5 | 65.5 | 58.05 | 62.9 | 67.5* | 65.5 |
| Benzoic | — | — | 2 | 2 | 3 | 4 | 4 | 4 | 4 |
| acid | | | | | | | | | |
| Sesame oil | — | 12.1 | 10.55 | 3.77 | 2.85 | 9.2 | 4.83 | — | 2.09 |
| q.s. to | | | | | | | | | |
| 100 ml | | | | | | | | | |

All values are in % w/v
*q.s. with castor oil to 100 ml

The solubility study results are presented in Table 5.

TABLE 5

| | Solubility |
|---|---|
| Formulation | (mg/g) |
| Test 10 | 104.6 |
| Test 11 | 111.8 |
| Test 13 | 121.2 |
| Test 14 | 113.5 |
| Test 15 | 102.4 |

Precipitation study result for oil layer is presented in Table 6.

TABLE 6

| Time | Reference | Test 13 |
|---|---|---|
| (Day) | % Fulvestrant | % Fulvestrant |
| 0 | 100 | 102 |
| 1 | 111 | 112 |
| 2 | 110 | 109 |
| 3 | 73 | 93 |
| 4 | 64 | 100 |
| 5 | 74 | 104 |
| 6 | 65 | 94 |
| 7 | 61 | 87 |
| 9 | 61 | 73 |
| 12 | 58 | 65 |

The test formulations were subjected to in-vivo study. 48 rats (Wister/Male) were distributed in four different groups (12 Rats each). Reference and test formulations were administered intramuscularly at a dose of 25 mg/kg. Blood samples were taken at specified period of time to analyze pharmacokinetic parameters (Cₘₐₓ, AUC₀₋₁, Tₘₐₓ). The results of in-vivo study are presented in Table 7.

TABLE 7
Pharmacokinetic parameters (mean ± S.D.)

| | Units | Reference | Test 13 | Test 14 | Test 15 |
|---|---|---|---|---|---|
| Cₘₐₓ | ng/mL | 19.63 ± 6.71 | 21.37 ± 5.85 | 17.24 ± 5.01 | 20.35 ± 4.80 |
| AUC₀₋ₜ | ng · hr/mL | 2463.56 ± 803.58 | 2614.69 ± 735.38 | 2340.90 ± 37.97 | 2652.42 ± 851.67 |
| *Tmax | hr | 48 (24-96) | 24 (9-48) | 24 (24-72) | 24 (9-144) |
| (Range) | | | | | |

*Median

In another aspect, the present specification provides formulation stability over time so that a product can be manufactured and introduced into the channels of commerce with sufficient dating as to be commercially reasonable. Generally, a stable product is one which when stored under the directed conditions retains at least 80% of label potency, e.g. at least about 90% of labeled potency at a designated date, which is generally at least one year, e.g. at least about 18 months, e.g. at least about 2 years, and e.g even longer. The stability data is presented in Table8 & 9.

TABLE 8
Assay (%)

| | |
|---|---|
| Test | 4 Weeks |

| formulations | Initial | 4° C.* | CRT** |
|---|---|---|---|
| 10 | 98.9 | 99.5 | 98.8 |
| 11 | 99.2 | 100.3 | 100.5 |
| 13 | 98.8 | 100.9 | 100.2 |
| 14 | 98.8 | 100.5 | 99.4 |
| 15 | 98.5 | 99.8 | 98.7 |

*product packed in 10 mL amber color vials
**(control room temperature) product packed in glass syringes (5 mL glass Schott prefillable syringe with Westar RS stopper)

TABLE 9

| | Initial | | | | | Refrigerated (4° C.)_4 Weeks* | | | | | CRT_4 Weeks** | | | | |
|---|---|---|---|---|---|---|---|---|---|---|---|---|---|---|---|
| Impurity | Test Formulation | | | | | | | | | | | | | | |
| Name | 10 | 11 | 13 | 14 | 15 | 10 | 11 | 13 | 14 | 15 | 10 | 11 | 13 | 14 | 15 |
| Unknown | ND | ND | ND | ND | ND | ND | ND | ND | ND | 0.03 | <0.05 | <0.05 | <0.05 | ND | <0.05 |
| Unknown | 0.06 | 0.06 | 0.06 | 0.06 | 0.06 | 0.06 | 0.06 | 0.06 | 0.06 | 0.06 | 0.07 | 0.06 | 0.06 | 0.06 | 0.06 |
| Unknown | ND | ND | ND | ND | ND | <0.05 | <0.05 | <0.05 | <0.05 | <0.05 | <0.05 | <0.05 | <0.05 | <0.05 | <0.05 |
| Unknown | 0.10 | 0.09 | 0.09 | 0.09 | 0.09 | 0.09 | 0.09 | 0.09 | 0.09 | 0.09 | 0.09 | 0.09 | 0.09 | 0.09 | 0.09 |
| Sulfone | 0.10 | 0.10 | 1.27 | 0.10 | 0.10 | 0.12 | 0.11 | 0.12 | 0.11 | 0.12 | 0.21 | 0.20 | 0.22 | 0.16 | 0.20 |
| Sterol | 0.10 | 0.10 | 1.85 | 0.10 | 0.09 | 0.10 | 0.10 | 0.09 | 0.09 | 0.10 | 0.11 | 0.09 | 0.09 | 0.10 | 0.09 |
| dimer | | | | | | | | | | | | | | | |
| Unknown | 0.07 | 0.07 | 2.39 | 0.07 | 0.07 | 0.09 | 0.08 | 0.08 | 0.08 | 0.08 | 0.09 | 0.08 | 0.08 | 0.10 | 0.09 |
| Unknown | ND | ND | 2.57 | ND | ND | <0.05 | <0.05 | <0.05 | <0.05 | <0.05 | <0.05 | <0.05 | <0.05 | <0.05 | <0.05 |
| Unknown | ND | ND | 2.67 | ND | ND | <0.05 | <0.05 | <0.05 | <0.05 | <0.05 | <0.05 | <0.05 | <0.05 | <0.05 | <0.05 |
| Unknown | ND | ND | 2.77 | ND | ND | <0.05 | <0.05 | ND | ND | <0.05 | <0.05 | ND | ND | <0.05 | ND |
| Total | 0.43 | 0.42 | 0.42 | 0.41 | 0.41 | 0.46 | 0.44 | 0.44 | 0.43 | 0.48 | 0.57 | 0.52 | 0.54 | 0.51 | 0.53 |
| Impurities | | | | | | | | | | | | | | | |

Sterol dimer - Process impurity, not monitored in the drug product;
Sulfone - Metabolite of Fulvestrant
*product packed in 10 mL amber color vials
**(control room temperature) product packed in glass syringes (5 mL glass Schott prefillable syringe with Westar RS stopper)

1. A pharmaceutical formulation comprising:
(i) fulvestrant or a pharmaceutically acceptable salt thereof;
(ii) benzoic acid;
(iii) one or more alcohols; and
(iv) one or more vegetable oils.

2. The pharmaceutical formulation as claimed in claim 1, wherein the vegetable oils are selected from one or more of castor oil, sesame oil, cottonseed oil, peanut oil, corn oil, soybean oil, olive oil or mixtures thereof.

3. The pharmaceutical formulation as claimed in claim 1, wherein the alcohols are selected from one or more of ethanol, benzyl alcohol or a mixture of ethanol and benzyl alcohol.

4. The pharmaceutical formulation as claimed in claim 1 comprising:
(i) 2-10% w/v fulvestrant or a pharmaceutically acceptable salt thereof;
(ii) 1-15% w/v benzoic acid;
(iii) 2-40% w/v of one or more alcohols; and
(iv) at least 55% w/v castor oil.

5. The pharmaceutical formulation as claimed in claim 4 comprising:
(i) 5% w/v fulvestrant or a pharmaceutically acceptable salt thereof;
(ii) 2-8% w/v benzoic acid;
(iii) 2-20% w/v benzyl alcohol;
(iv) 2-20% w/v ethanol; and
(iv) at least 55% w/v castor oil.

6. The pharmaceutical formulation as claimed in claim 5 comprising:
(i) 5% w/v fulvestrant or a pharmaceutically acceptable salt thereof;
(ii) 4% w/v benzoic acid;
(iii) 10% w/v benzyl alcohol;
(iv) 10% w/v ethanol; and
(iv) 55-70% w/v castor oil.

7. The pharmaceutical formulation as claimed in claim 1 comprising:
(i) 5% w/v fulvestrant or a pharmaceutically acceptable salt thereof;
(ii) 2-8% w/v benzoic acid;
(iii) 2-20% w/v benzyl alcohol;
(iv) 2-20% w/v ethanol;
(v) 55-70% w/v castor oil; and
(vi) 2-12% w/v sesame oil.

8. The pharmaceutical formulation as claimed in claim 7 comprising:
(i) 5% w/v fulvestrant or a pharmaceutically acceptable salt thereof;
(ii) 4% w/v benzoic acid;
(iii) 10% w/v benzyl alcohol;
(iv) 10% w/v ethanol;
(v) 55-70% w/v castor oil; and
(vi) 2-12% w/v sesame oil.

9. A method of treating a hormone receptor positive tumor by administration to a human in need of such treatment an intra-muscular injection of a pharmaceutical formulation claimed in claim 1, whereby a therapeutically significant blood plasma fulvestrant concentration of at least 2.5 ng/ml is attained for at least 2 weeks after injection.

10. The method as claimed in claim 9, wherein the blood plasma fulvestrant concentration is attained for at least 4 weeks after injection.
